(19) United States
(12) Patent Application Publication
(10) Pub. No.: US 2022/0087330 A1
(43) Pub. Date: Mar. 24, 2022

(54) A BRA REDUCING SECONDARY CANCER RISK

(71) Applicant: HACETTEPE UNIVERSITESI, Ankara (TR)

(72) Inventors: Ferah YILDIZ, Ankara (TR); Nur KODALOGLU, Ankara (TR)

(73) Assignee: HACETTEPE UNIVERSITESI, Ankara (unknown)

(21) Appl. No.: 17/423,947

(22) PCT Filed: Feb. 28, 2020
(86) PCT No.: PCT/TR2020/050159

§ 371 Date: Jul. 19, 2021

(30) Foreign Application Priority Data
Mar. 1, 2019 (TR) 2019/03135

(51) Int. Cl. A41C 3/00; A61N 5/10; G21F 1/08; G21F 3/02

(52) U.S. Cl. CPC A41C 3/0064; A61N 5/10; A61N 2005/1094; G21F 3/02; G21F 1/08

(57) ABSTRACT

The present invention relates to a bra which is used for the purpose of reducing secondary cancer risk during treatment of breast cancer patients

### TECHNICAL FIELD

The present invention relates to a bra which is used for the purpose of reducing secondary cancer risk during treatment of breast cancer patients.

### BACKGROUND OF THE INVENTION

Computed tomography images of patients who will start radiotherapy treatment are required in order to perform drawings (contouring) of tumors and organs aimed to be protected around the tumor. After patients have computed tomography scan, their images are sent to a radiation oncologist regardless of their diagnosis. Radiation oncologist performs contouring of tumor and critical organs (organs aimed to be protected by receiving less doses around the tumor) in computed tomography images slice by slice. Examples such as heart, lung, opposite breast (contralateral breast) can be given as these organs for breast cancer. Following the contouring process, a treatment plan is done and the patient diagnosed with breast cancer receives radiotherapy for approximately 25 working days according to this plan. The most important point during these treatments is to provide the same position as in the computed tomography whereby the patient was scanned on the first day in order to ensure that both the tumor can take the planned dose in the same place and critical organs aimed to be protected do not take more doses than the planned dose. Therefore, Image Guided Radiotherapy (IGRT) is performed (i.e. controlling the position of the patient by receiving the related images before treatment). Just at this point, use of a classic-style bra for the breast which is an organ with high mobility while trying to maintain the same position in the order of millimeter every day is not successful enough to provide the same position. Concepts such as breast shapes, sizes, and sagging vary from patient to patient and the possibility of getting into different shapes in any cup shaped bra is an undesirable situation. Therefore, there is need for a new bra which has a cavity that will not cause a breast with tumor to be pushed to any direction by releasing it completely, and will reduce the out-of-field dose by removing pushing away the non-cancerous breast from the breast to be treated as well, will eliminate the need for producing different bras according to the breast size (A,B,C,D cup, etc.) of each patient, and can be used for both breasts, instead of cap-shaped bras known in the classical sense.

The Chinese utility model document no. CN201444932, an application in the state of the art, discloses a bra with a radiation-protective function. The bra comprises a side band, a cup, a shoulder strap and a hook which are located at the end of the side bands and enable interconnection. The cup consists of two fabric layers and a protective layer placed between them. A magnetic fabric layer is located behind the inner fabric layer of the cup. The protective layer has a protection feature against electromagnetic radiation. The protective layer is covered with silver fiber and nylon filament. While the number of breast cancer patients is increasing day by day, the bras are available for preventing electromagnetic radiation from damaging women's breast health.

The United States patent document no. U.S. Pat. No. 7,902,530, an application in the state of the art, discloses preventing damaging healthy tissues in tumor treatment by using Cerrobend and reducing the risk to develop secondary malignancies after radiation therapy. Blocks made of melted Cerrobend are used for performing treatment upon being prepared in special forms to exclude the normal tissue around the tumor. Custom melted and shaped Cerrobend blocks enable to reduce secondary cancer risk in patients after radiation therapy.

### SUMMARY OF THE INVENTION

An objective of the present invention is to realize a bra which enables to reduce secondary cancer risk during radiotherapy treatment of breast cancer patients.

Another objective of the present invention is to realize a bra which enables to keep the non-cancerous breast out of the area in order to avoid receiving radiation in the treatment of breast cancer unnecessarily.

Another objective of the present invention is to realize a bra which can be used together with the shield that reduces the dose arising from the radiation being scattered during treatment.

Another objective of the present invention is to realize a bra which can be positioned easily and correctly despite the shield used for reducing the radiation dose, has a simple and flexible structure, and can adapt to irregularly shaped treatment areas.

Another objective of the present invention is to realize a bra which can adapt to the breast size of each patient during radiotherapy treatment and can be used for both breasts.

### DETAILED DESCRIPTION OF THE INVENTION

“A Bra Reducing Secondary Cancer Risk” realized to fulfil the objectives of the present invention is shown in the figures attached, in which:

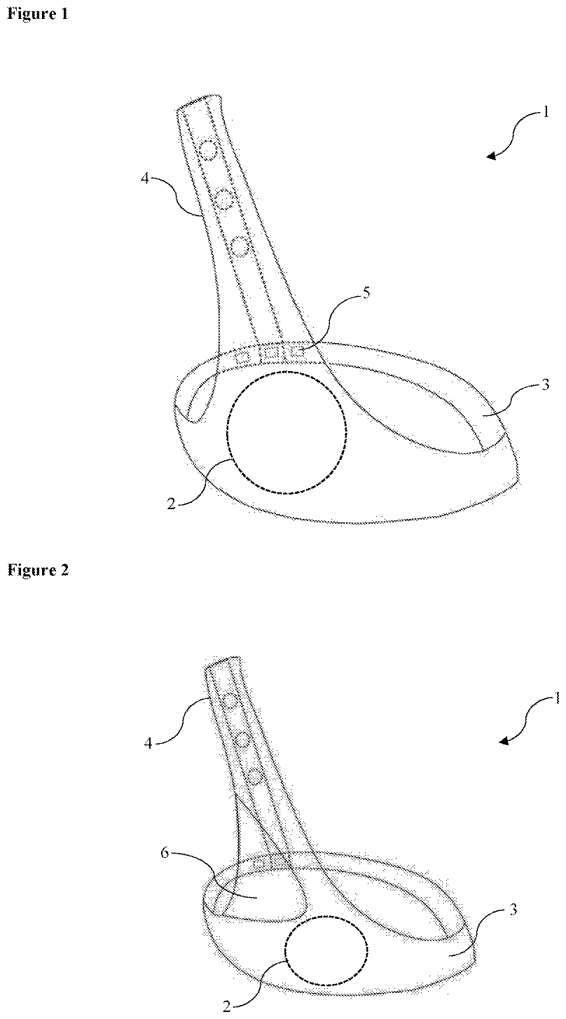

FIG. 1 is a front view of the inventive bra.

FIG. 2 is a view of the inventive bra wherein a pushing element is added.

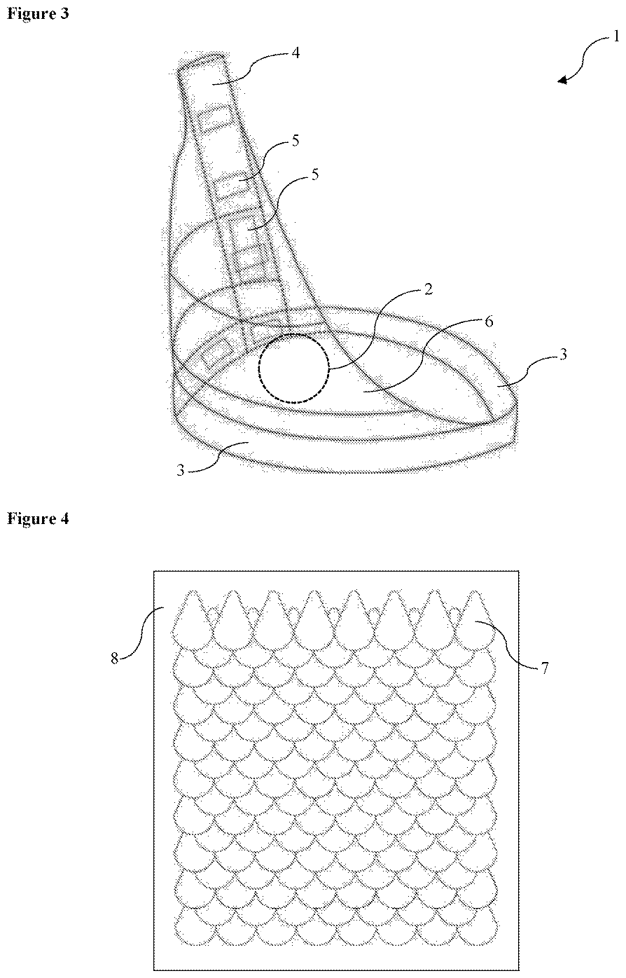

FIG. 3 is another view of the inventive bra wherein a pushing element is added.

FIG. 4 is a view wherein the inventive shields are individually sewn to the holder.

The components illustrated in the figures are individually numbered, where the numbers refer to the following:
1. Bra
2. Retaining element
3. Side band
4. Shoulder strap
5. Connection element
6. Pushing element
7. Shield
8. Holder

The inventive bra (1) reducing secondary cancer risk during treatment of breast cancer patients comprises:
a retaining element (2) which retains only the non-cancerous breast to protect it by leaving the cancerous breast exposed, at the step of administering radiation therapy to the cancerous breast;
at least one side band (3) which extends from the side parts of the retaining element (2) towards the patient's back in order to ensure that the retaining element (2) holds on to the body;
at least one shoulder strap (4) which extends from the retaining element (2) up to the side band (3) on the back part by passing over the shoulder on the side where the non-cancerous breast is located, secures retaining the breast by enabling the retaining element (2) to press the non-cancerous breast, and has an adjustable structure;
at least one connection element (5) which is located on the end of the side bands (3) and the shoulder strap (4) and enables to interconnect the side bands (3) and the shoulder strap (4);
a pushing element (6) one end of which is sewn to the retaining element (2) point between two breasts, other end of which is fixed to the connection element (5) on the patient's back by passing over the retaining element (2) and under the patient's arm;
at least one shield (7) which protects the non-cancerous breast from the radiation being scattered inside the treatment room, upon being placed onto the retaining element (2) afterwards; and
at least one holder (8) which ensures that the shields stay together on the retaining element (2) in a desired shape and number.

In the inventive bra (1), the retaining element (2) is one in number. The retaining element (2) is placed onto the breast such that it will cover the non-cancerous breast and does not contact the cancerous breast in no way. Thereby, it is ensured that the non-cancerous breast is removed from the area wherein treatment will be administered and it is protected from the radiation scattering around for treatment, while administering radiotherapy treatment to the cancerous breast. The shields (7) fixed onto the holder (8) during radiotherapy treatment and used for protection are placed onto the retaining element (2). The retaining element (2) is made of a sponge-like soft fabric which is flexible and can be used directly on the patient's skin and non-toxic. Thereby, it creates convenience in terms of use in every patient and eliminates the need for using additional material preventing irritation between skin and bra (1) or any damage that may occur, by being made of a sponge-like soft fabric which is flexible and can be used directly on the patient's skin and non-toxic. At the same time, the retaining element (2) is produced such that it will adapt to all breast structures in different sizes (A, B, C, D . . . ) and in one size and it avoids the need for producing the retaining element (2) separately and the expenditures made, by being used for both the right and the left breast.

The side band (3) included in the inventive bra (1) is connected from the side parts of the retaining element (2) located in front of the non-cancerous breast ant it extends towards the back by passing under the right and left arm in order to ensure that the retaining element (2) is fixed to the body. In the meantime, it does not contact the cancerous breast and create any effect that will damage its natural posture (sagging, shape). The side band (3) has a flexible band that holds on to the connection element (5) from its end part and enables to join it such that it will fit the size of the patient in the back region. By means of the band, it is possible to administer radiotherapy treatment to patients having different sizes by using the same bra (1) by means of the band. The side band (3) is made of a sponge-like soft fabric which is flexible and can be used directly on the patient's skin. Therefore, it can be conveniently used without creating skin sensitivity in patients.

The shoulder strap (4) included in the inventive bra (1) is connected with the retaining element (2) from its upper part and it extends towards the patient's back over the shoulder on the side where the non-cancerous breast is located. The shoulder strap (4) is made of a flexible material. Thereby, it can be easily adjusted according to the patient's size. Band is located on the end of the shoulder strap (4) and there are holes at regular intervals on the band (FIGS. 1 and 2). The holes ensure that the threads at the ends of adhesive silicone parts required for treatment and to be adhered onto the non-cancerous breast, are passed and joined. Like the retaining element (2) and the side band (3), the shoulder strap (4) is made of a sponge-like soft fabric which is flexible and can be used directly on the patient's skin and it does not cause any irritation on the patient's skin.

The connection element (5) included in the inventive bra (1) is connected with the shoulder strap (4) and the end of the side straps (3) extending under the right and left arm and it enables to interconnect the shoulder strap (4) and the side straps (3) in the back part of the patient. Also, the connection element (5) ensures fixation to the back region of the patient after the pushing element (6) enables removal of the breast from the treatment area by pressing the retaining element (2) upon passing over the retaining element (2). The connection element (5) is an element such as hook, Velcro, hook-and-eye fastener providing connection.

The pushing element (6) included in the inventive bra (1) is sewn onto the retaining element (2) from its one end in order to provide easier control in large-size breasts and apply the same pushing effect to the breast during all days when the patient receives treatment and its other end extends to the connection element (5). The pushing element (6) enables to remove the large-size breast from the cancerous breast during the treatment and supports the retaining element (2) (FIG. 3). In another embodiment of the invention, the pushing element (6) is sewn into the aperture between the side band (3) and the shoulder strap (4) (FIG. 2). In this embodiment, the pushing element (6) helps to remove the small-size breast from the cancerous breast receiving treatment.

The shield (7) which is used together with the inventive bra (1) can be fixed onto the holder (8) at a desired interval. Sepiolite, copper, lead, barium borate, borax and Cerrobend materials having the capacity to stop radiation can be used in production of the shield (7). Although lead has the highest capacity to stop radiation among these materials, Cerrobend materials having the highest availability and price-cost analysis is preferred. The Cerrobend material is made of 50% bismuth, 26.7% lead, 13.3% tin and 10% cadmium composition. This material can be melted at 70° C. without needing to reach high temperatures to be shaped. After melting is carried out at this temperature, the Cerrobend material is poured into silicone molds in the form of water drops or bars and a plurality of shields (7) are produced in small sizes. Then, the shield (7) is placed onto the retaining element (2) at an interval to ensure that the non-cancerous breast is subjected to a radiation dose as little as possible and such that it will be partially overlapping. Thickness of the shield (7) is preferably in the range of 4 to 7 mm. the shield (7) can be in full contact with the skin after being placed onto the retaining element (2) upon being fixed on the holder (8), by means of its small structure: it reduces the rate of radiation, scattering inside the treatment room, to reach the skin by means of its full retaining; enables to adapt to irregularly-shaped treatment areas easily and accurately as well. In the invention, the shield (7) protects the breast from the radiation composing the out-of-field dose scattering inside the treatment room and/or from the linear accelerator device.

In another embodiment of invention, the shield (7) is also produced in thyroid and pediatric forms as well.

The holder (8) being used together with the inventive bra (1) creates an area in order to place the shield (7) at a desired interval and to desired locations and it has a size suitable to provide this. The shields (7) to be placed onto the holder (8) can be sewn individually. In individual sewing method, considering the time when the shields (7) are sewn to the holder (8), production of the holder (8) with the shield (7) can be easily realized by adhering the shields (7) to strip-shaped velvet pieces that will create a soft feeling on the patient's skin and sewing these strips to the holder (8) on top of each other. Then, the holder (8) is fixed onto the retaining element (2) by means of Velcro; secondary cancer risk is decreased by reducing the dose of radiation scattering out of the treatment area and to be taken into the body unnecessarily. The holder (8) is produced by using a fabric or a material having the ability to move and retain like fabric.

The inventive bra (1) enables to keep the breast with high mobility in the same position in every day and to administer treatment while s/he is in the same position, when the patient has a computed tomography during the treatment period by means of its configuration. In addition, by means of the Cerrobend shield (7) placed onto the retaining element (2), it can be used in any patient easily and without damaging the form of the cancerous breast at all regardless of the fact that cancerous breasts of breast cancer patients are right or left and their dimension during the radiotherapy treatments. Thereby, risk of secondary cancer arising from radiation being scattered into the treatment room is reduced.

Within these basic concepts; it is possible to develop various embodiments of the inventive bra (1); the invention cannot be limited to examples disclosed herein and it is essentially according to claims.

1. The inventive bra (1) reducing secondary cancer risk during treatment of breast cancer patients comprising:
a retaining element (2) which retains only the non-cancerous breast to protect it by leaving the cancerous breast exposed, at the step of administering radiation therapy to the cancerous breast;
at least one side band (3) which extends from the side parts of the retaining element (2) towards the patient's back in order to ensure that the retaining element (2) holds on to the body;
at least one shoulder strap (4) which extends from the retaining element (2) up to the side band (3) on the back part by passing over the shoulder on the side where the non-cancerous breast is located, secures retaining the breast by enabling the retaining element (2) to press the non-cancerous breast, and has an adjustable structure;
at least one connection element (5) which is located on the end of the side bands (3) and the shoulder strap (4) and enables to interconnect the side bands (3) and the shoulder strap (4);
characterized by
a pushing element (6) one end of which is sewn to the retaining element (2) point between two breasts, other end of which is fixed to the connection element (5) on the patient's back by passing over the retaining element (2) and under the patient's arm;
at least one shield (7) which protects the non-cancerous breast from the radiation being scattered inside the treatment room, upon being placed onto the retaining element (2) afterwards; and
at least one holder (8) which ensures that the shields stay together on the retaining element (2) in a desired shape and number.

2. A bra (1) according to claim 1; characterized by the retaining element (2) which is placed onto the breast such that it will cover the non-cancerous breast and ensures that the non-cancerous breast is removed from the area wherein treatment will be administered and it is protected from the radiation scattering around for treatment, while administering radiotherapy treatment to the cancerous breast by not contacting the cancerous breast in any way.

3. A bra (1) according to claim 1, characterized by the retaining element (2) whereon the shields (7) fixed onto the holder (8) during radiotherapy treatment and used for protection are placed onto the retaining element (2).

4. A bra (1) according claim 1, characterized by the retaining element (2) which creates convenience in terms of use in every patient and eliminates the need for using additional material preventing irritation between skin and bra or any damage that may occur, by being made of a sponge-like soft fabric which is flexible and can be used directly on the patient's skin and non-toxic.

5. A bra (1) according claim 1, characterized by the retaining element (2) which is produced such that it will adapt to all breast structures in different sizes (A, B, C, D . . . ) and in one size and it enables to avoid the need for producing the retaining element (2) separately and the expenditures made, by being used for both the right and the left breast.

6. A bra (1) according claim 1, characterized by the side band (3) which is connected from the side parts of the retaining element (2) located in front of the non-cancerous breast ant it extends towards the back by passing under the right and left arm in order to ensure that the retaining element (2) is fixed to the body.

7. A bra (1) according claim 1, characterized by the side band (3) which does not contact the cancerous breast and create any effect that will damage its natural posture (sagging, shape).

8. A bra (1) according claim 1, characterized by the side band (3) which has a flexible band that holds on to the connection element (5) from its end part and enables to join it such that it will fit the size of the patient in the back region.

9. A bra (1) according claim 1, characterized by the side band (3) which makes is possible to administer radiotherapy treatment to patients having different sizes by using the same bra (1) by means of the band.

10. A bra (1) according claim 1, characterized by the side band (3) which is made of a sponge-like soft fabric that is flexible and can be used directly on the patient's skin.

11. A bra (1) according claim 1, characterized by the shoulder strap (4) which is connected with the retaining element (2) from its upper part and it extends towards the patient's back over the shoulder on the side where the non-cancerous breast is located.

12. A bra (1) according claim 1, characterized by the shoulder strap (4) which can be easily adjusted according to the patient's size due to the fact that it is made of a flexible material.

13. A bra (1) according claim 1, characterized by the shoulder strap (4) wherein band is located on its end and there are holes at regular intervals on the band.

14. A bra (1) according claim 1, characterized by the shoulder strap (4) which has holes ensuring that the threads at the ends of adhesive silicone parts required for treatment and to be adhered onto the non-cancerous breast, are passed and joined.

15. A bra (1) according claim 1, characterized by the shoulder strap (4) which is made of a sponge-like soft fabric which is flexible and can be used directly on the patient's skin and it does not cause any irritation on the patient's skin.

16. A bra (1) according claim 1, characterized by the connection element (5) which is connected with the shoulder strap (4) and the end of the side straps (3) extending under the right and left arm and enables to interconnect the shoulder strap (4) and the side straps (3) in the back part of the patient.

17. A bra (1) according claim 1, characterized by the connection element (5) which ensures fixation to the back region of the patient after the pushing element (6) enables removal of the breast from the treatment area by pressing the retaining element (2) upon passing over the retaining element (2).

18. A bra (1) according claim 1, characterized by the connection element (5) which is at least one of elements such as hook, Velcro, hook-and-eye fastener providing connection.

19. A bra (1) according claim 1, characterized by the pushing element (6) which is sewn onto the retaining element (2) from its one end in order to provide easier control in large-size breasts and apply the same pushing effect to the breast during all days when the patient receives treatment and its other end extends to the connection element (5).

20. A bra (1) according to claim 1, characterized by the pushing element (6) which is sewn into the aperture between the side band (3) and the shoulder strap (4) so as to be used in small-size breasts.

21. A bra (1) according claim 1, characterized by the shield (7) which can be fixed onto the holder (8) at a desired interval.

22. A bra (1) according claim 1, characterized by the shield (7) which is made of Cerrobend material.

23. A bra (1) according to claim 22, characterized by the shield (7) which is made of Cerrobend having 50% bismuth, 26.7% lead, 13.3% tin and 10% cadmium composition.

24. A bra (1) according claim 1, characterized by the shield (7) which is in the form of water drop or bar.

25. A bra (1) according claim 1, characterized by the shield (7) which is made upon being poured into silicone molds.

26. A bra (1) according claim 1, characterized by the shield (7) which is placed onto the retaining element (2) at an interval to ensure that the non-cancerous breast is subjected to a radiation dose as little as possible and such that it will be partially overlapping, after being produced.

27. A bra (1) according claim 1, characterized by the shield (7) which thickness of which is preferably in the range of 4 to 7 mm.

28. A bra (1) according claim 1, characterized by the holder (8) which has a size suitable to create an area in order to place the shield (7) at a desired interval and to desired locations.

29. A bra (1) according claim 1, characterized by the holder (8) which is fixed onto the retaining element (2) by Velcro.

30. A bra (1) according claim 1, characterized by the holder (8) which is produced by using a fabric or a material having the ability to move and retain like fabric.
